(19) United States
(12) Patent Application Publication
(10) Pub. No.: US 2003/0108968 A1
(43) Pub. Date: Jun. 12, 2003

(54) Method for detecting the reactivity of lymphocyte in blood to specific antigen

(76) Inventor: Jun Hu, Shanxi (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/220,798

(22) PCT Filed: Jan. 18, 2001
(86) PCT No.: PCT/CN01/00064

(30) Foreign Application Priority Data
Mar. 1, 2000 (CN) 00113747.6

(51) Int. Cl. G01N 33/53

(52) U.S. Cl. 435/7.24

(57) ABSTRACT

The present invention discloses a method for detecting the reactivity of lymphocyte in blood to a specific antigen, wherein the standard cell strains expressing a known HLA antigen respectively is cultured with the recipient's lymphocytes for 2-28 hours in vitro, and then the reactivity of the lymphocytes is determined through an immunological assay. The invention provides a method to monitor dynamically the recipient's immune status to donor organ after transplantation, and also provide a basis for adjusting the clinical immunosuppressant dosage administrated to the recipient, which can improve the success rate of organ transplantation significantly.

### FIELD OF THE INVENTION

The present invention relates to a method for detecting the reactivity of lymphocyte in blood to some specific antigens in order to determine the extent of rejection of the recipient towards donor organ after organ transplantation. By using standard cell strain, the killing function and the proliferative response ability of T-lymphocytes in the peripheral blood of the recipients are evaluated in vitro so as to determine the rejection status of the recipients towards the transplanted organ, thereby providing a basis of reference for clinical practice.

### BACKGROUND OF THE INVENTION

The main factor for organ transplant rejection lies in the differences of antigens between the recipient and the donor. In allogenic transplantation, the antigens that cause rejection reactions are specific and limited in number, wherein the main factor causing rejection lies in the difference of human leukocyte antigen (HLA). By 1991, 161 HLA antigens had been identified in the whole human population, among which, there were 122 Type I antigens, and 39 Type II antigens. Mixed lymphocyte reactions are mainly induced by Type II antigens, and cytotoxicity T lymphocytes (CTL) killing activity is mainly induced by Type I antigens (Transplantation Immunology, ed. by Chen Shi, Hu Bei Science and Technology Press, China, October, 1998). These HLA antigens can be obtained by screening from the human population or utilizing genetic engineering to develop, that is, to establish the standard cell strains which possess known genetic background and express a specific HLA or several HLA antigens stably. These standard cell strains are characterized in that the cell of each standard cell strain will only express a specific antigen or several antigens among HLA antigens. For example, on the cell membrane surface of a kind of standard cell strain, only HLA-A23 is expressed, but on that of another kind of standard cell strain, only DR9 is expressed (more than a dozen HLA antigens being expressed on B cell of the normal human beings). In this way, one can establish many different standard cell strains that express only one or several HLA antigens respectively. At present, Lamda and Pel-freeze, two American companies, have established standard cell strains that express several HLA antigens on the surface of one cell, and have placed those established standard cell strains on the market as a merchandise called PRA Assay Plate. This product is used mainly as the antigen carrier to determine the recipient's antibody level and specificity against HLA before organ transplantation, and thereby predicting the possibility of hyper-acute rejection and providing basis for the selection of donor. However, heretofore, the cell strains that express only one Type I HLA or one Type II HLA have not been established.

Mixed lymphocyte reaction has been established for several decades, and in immunology, they are used in the methods for determining the cellular immunity status of the body to certain specific antigen or several specific antigens. In the field of organ transplantation, it was previously used to determine the matching status of HLA antigens between the recipient and the donor before transplantation so as to predict the surviving quality of the transplanted organ and the possibility of rejection. But, because of the complicated procedure, poor stability and repeatability as well as time-consuming (usually, 5-7 days were needed for obtaining the results), it is difficult for the reaction to be applied to the case that the organ-donor is a corpus. Thus, after other better HLA typing technologies developed, this method has been gradually eliminated (Cellular & Molecular Immunology, ed. by Boquan Jin, World Books Publishing Co., August, 1995, p230).

Pre-processing Lymphocyte Typing Test (Cellular & Molecular Immunology, p230): to overcome the shortcomings of the mixed lymphocyte reactions in typing, one developed this method. This method comprises the following steps: cells that are known to contain a specific HLA antigen or several specific HLA antigens are taken as irritation cells, and T-lymphocytes that don't contain the specific HLA antigens are isolated as reaction cells. Then, the irritation cells and the reaction cells are mixed together for mixed lymphocyte reaction. After culturing for 9-14 days, the proliferation of the reaction cells gradually stops. Finally, the reaction cells become memory cells that are sensitized by a specific HLA or several specific HLA antigens. They are termed pre-processed (pre-sensitized) cells, and are stored in liquid nitrogen. While match-typing, these cells are thawed and are used as reaction cells, and the lymphocytes of the recipient or donor are used as irritation cells. When these irritation cells contain the same HLA antigens as those contained in the irritation cells used in the pre-procession, the pre-processed reaction cells will immediately manifest a memory reaction, and during 20-30 hours, proliferation reaction will rapidly take place. In this way, it can determine whether the lymphocytes of the recipient or donor contain this specific antigen.

Cytotoxicity T-lymphocyte killer test has also been established for several decades. In immunology, it has often been used to determine the sensitization status of the body to certain antigen or several antigens existing on the surface of the cells (Immunology and Immunoassay, ed. by Yixun Tao, the People's Health Publishing House, China, October, 1989, p250). In recent years, the detection of the killer cell activity has also been used in monitoring the rejection reaction in the organ transplantation. However, because of the differences among individuals, the application of this test so far has been limited to one-to-one analysis on the cells from the donor as the target cells. But only the living donor for organ transplantation can provides the target cells for the detection constantly. For this reason, so far, this method has only been used in the detection of the organ transplants provided by living donors, otherwise there is no source of target cells. So, it is difficult to use this method widely.

At present, the number of the patients to receive organ transplantation increases by more than 40,000 cases every year. After organ transplantation, the recipient has to take immunosuppressant for a long time. If the dose is too high, it not only increase cost, but also promote the possibility of recipient suffering infectious diseases and malignant tumors. If the dose is too low, the transplanted organ will be rejected and functions thereof will be lost. How to select the proper dose for each specific individual has troubled the clinical practitioner for a long time. For years, the means of biopsy and paracentesis has been used as the “golden” index for guiding the administration, but because of its' expensive cost and suffering, it is not easy for it to be accepted by the patients. Furthermore, paracentesis is quite risky itself, and it is not advisable to conduct it frequently (Transplantation Immunology, p200). Therefore, many patients die suddenly after organ transplantation without the doctor's awareness because of the rejection reaction that causes the transplanted organ to lose the function thereof.

### SUMMARY OF THE INVENTION

The present invention is to provide a method for determining the reactivity of the lymphocytes in blood to a specific antigen(s). In this method, the “specific antigen(s)” means the donor antigen that is able to cause rejection reactions in the recipient's body, and the “reactivity to the specific antigens” refers to the reaction status of a recipient's lymphocytes towards a specific antigen(s).

The present invention provides a method for determining the reactivity of the lymphocytes in blood to a specific antigen(s), which is characterized by the following steps: the T-lymphocytes in the peripheral blood of the recipient are prepared into cell suspension with cell culture medium, and then by using a cell assay plate prepared (coated) with the standard cell strain containing the known HLA antigens, the cell suspension is added to each well of the cell assay plate in the target to effector ratio 1:0.5-10, and then the cells are cultured for 2-28 hours; The difference between the reactivity of the recipient's lymphocytes in the testing wells containing donor antigens and reactivity of the recipient's lymphocytes in the control wells without donor antigen was distinguished, and thereby, whether the recipient's lymphocytes are sensitized can be determined.

### DETAILED DESCRIPTION OF THE INVENTION

The present invention provides a method for determining the reactivity of the T-lymphocytes in blood to a specific antigen(s) after organ transplantation, which provides a means to monitor dynamically the recipient's immune status to donor organ by detecting T-lymphocytes in peripheral blood of the recipient, and thereby provides a basis for adjusting the clinical immunosuppressant dosage administrated to the recipient and can be used to predict the occurrence of rejection reaction before the clinical symptoms are present. Moreover, It also possesses the advantages of painless detection, shorter time, lower cost, higher specificity, and higher reliability.

The present invention provides a method for determining whether there is rejection reaction in the recipient's body through a mixed lymphocytes reaction in vitro of the T-lymphocytes in the recipient's peripheral blood with the standard cell strain containing known HLA antigens. The method is characterized by the following steps: the T-lymphocytes in the peripheral blood of the recipient are prepared into cell suspension with cell culture medium, and then by using a cell assay plate prepared with standard cell strains containing known HLA antigens, the cell suspension is added to each well of the cell assay plate in the target-effector ratio 1:0.5-10, and then the cells are cultured for 2-28 hours; and thereafter, the reactivity of the lymphocytes is detected by an immunological assay.

In the said detection, a microscope can be used to observe whether cell transformation takes place in each well, and its transformation rate can be obtained through cell counts so as to determine whether there is rejection reaction in the body of the recipient. The said culture medium can be a serum-free culture medium with labeled thymine triphosphate(TTP). During the 6-28 hours for cell culture, the labeled TTP is incorporated in intracellular DNA, which make the cell nucleus presenting certain significant feature under common microscope or fluorescence microscope.

In this type of mixed lymphocyte reactions wherein the standard cell strains containing various Type I and Type II HLA antigens are used as the irritation cells, and the recipient's lymphocytes are used as pre-sensitized reaction cells, only if the reaction cells are indeed sensitized in the body can they present cell transformation and proliferation rapidly when meeting the donor antigens in vitro, that is to say, the reaction cells are transformed into lymphoblast. The transformed cells possess the following features: larger in size, increased cytoplasm, vacuoles present, clear nucleole, and loose chromatin, all which make them distinguishable easily in morphology. If the labeled TTP has been added to the culture medium, then the morphological identification of the cell nucleus is even easier. Because the standard cell strain in each well of the cell assay plate contains certain or several specific HLA antigen(s), the type of donor antigens causing rejection reaction can be determined based on the position of the well where cell transformation (or proliferation) occurs. The transformation rate can be obtained after counting the transformed cells in each well. Then, the degree of rejection reaction in the body can be determined by the value which is obtained by subtracting the average transformation rate of all the wells without donor antigens from that of the wells containing donor antigens. Although this morphological observation method is convenient and economical, and gives out the results quickly, the man-mage factors are almost unavoidable, and what's more, the workload for counting cell is quite great. So, some instruments can also be used in the present invention for eliminating man-made factors and reducing workload.

The present method can also be proceeded as follows. After cell culture for 2-6 hours as above described, the same amount of the supernatant in each well is removed into the corresponding culture well of a blank cell assay plate, respectively, in order to carry out enzymatic reaction for determining activity of dehydrogenase. After the reaction is stopped, the wavelength at which the maximum absorption takes place is selected in the wavelength range of 480 nm-630 nm, and the light absorption value of the solution in each well is read at the selected wavelength. From these absorption values, the rejection reaction level in the body of the recipient can be determined. Dehydrogenase is one of the enzymes present in the cells. Normally, it cannot get through the cell membrane. However, when the target cells (the standard strain) containing donor antigens are damaged due to the challenge from the sensitized effector cells (T-lymphocytes), the permeability of the cell membrane is changed and the dehydrogenase can be released into the medium. Therefore, the content of the dehydrogenase in each well can be determined through enzymatic reaction, and the number of cells killed or damaged can be deduced therefrom. In this method, two control groups should be established: the natural release control group, and the maximum release control group, and 4-10 culture wells without donor antigens are used as control wells. Because the recipient has to undergo a lot of examinations before and after organ transplantation, the approximate scope of the types of HLA antigens causing rejection reaction can be determined substantially based on the data obtained from the previous examinations. Thereby, the positions of the culture wells in which donor antigens should or should not be contained are available. The value obtained by subtracting the average absorption value of the natural release control group from that of the wells containing donor antigens is divided by the value obtained by subtracting the average absorption value of the natural release group from that of the maximum release control group. The resultant quotient can indicate the rejection reaction level in the body of the recipient, the higher the quotient, the more the killer cells, and the more intensive the rejection reaction.

The present method can also be proceeded as follows. After the cell culture for 6-16 hours as described above, to each well of the cell assay plate, a same amount of 3-(4,5-dimethylthiazol-2-yl)-2,5-diphenyltertra-zoliumbromide (MTT) solution is added, and the cell culture is continued for 2-6 hours, at which the cell transformation status can be determined by detecting the residual amount of MTT in the supernatant or determining the amount of MTT transformation in the transformed cells. 1) The supernatant in each well is harvested, and within the wavelength range of 480 nm-630 nm, the wavelength with the maximum absorption of the solution takes place is selected, and the light absorption value of the solution in each well is read at the selected wavelength. Because this method is used to determine residual MTT amount in the culture medium, and the wells with high absorption values have no cell proliferation, which should be taken as the control wells, while the antigens contained in the wells with lower absorption values shall be considered as donor antigens, which cause rejection reaction in the body of the recipients. From the average absorption value of the control wells, the average absorption value of the wells containing donor antigens is subtracted. The higher the resultant difference is, the more active the proliferation of the recipient's lymphocytes is. It suggests that there is rejection reaction in the recipient's body. 2) To each well of the cell plate in which the supernatant has been removed, the same amount of DMSO is added so that formazane crystals will be dissolved out of the transformed cells. Within the wavelength range of 480 nm-630 nm, the wavelength with the maximum absorption value is selected, and the light absorption value of the supernatant in each well is read at the selected wavelength, respectively. Because this method is used to determine formazane crystals derived from MTT in transformed cells, the wells with low light absorption value have no proliferation reaction, and are considered as natural control wells, while the antigens contained in those wells with higher light absorption value are the donor antigens, which will cause rejection reaction in the body of the recipients. From the average light absorption value of the wells with donor antigens, the average light absorption value of the control wells is subtracted. The higher the resultant difference is, the more active the proliferation of the recipient's lymphocytes is. This suggests that there is rejection reaction in the body of the recipient.

The present method can also be proceeded as follows. When the standard cell strain on the cell assay plate is prepared, it can be treated by MTT so that each cell contains the same amount of formazane crystals from the reduction of MTT. When the cell assay plate is involved in cell culture according to the above mentioned mixed lymphocyte reaction, it is also a killer test process using the standard cell strain as the target cells. Thus, the detection results can be obtained by determining the activity of the killer cells. Therefore, the present invention can also determine if there will be rejection reaction in the body of the recipient by determining the killer cell activity using MTT assay. In the killer cell test, the wells containing donor antigens are used as the test reaction wells, while the ones without donor antigens are used as control wells for determining the activity of the killer cells. There are three methods for this purpose.

After the cells are cultured for 3-8 hours, the same amount of oxidizing agent is added in each well of the cell assay plate. The culture continues for 1-4 hours. The oxidizing agent can oxidize formazane crystals in the damaged target cells into MTT. After the cell culture is stopped, the wavelength at which the maximum absorption value takes place is selected within the wavelength range of 480 nm-630 nm, and the light absorption value of the supernatant in each well is read at the selected wavelength. Because this method is used to determine the content of formazane in the damaged cells, the higher light absorption values of a well is, the greater content of formazane therein is, the more cells are damaged in this well and it indicates that the antigens contained therein are donor antigens, which cause rejection reaction in the body of the recipient. The average absorption value of the wells without donor antigens is subtracted from that of the wells with donor antigens. The higher the resultant difference is, the more intensive the rejection reaction is.

After the cells are cultured for 4-10 hours, the supernatant with the oxidizing agent is removed. A same amount of DMSO solution is added to each well to make formazane crystals in intact target cells dissolve in DMSO. The supernatant in each well is harvested, respectively, and within the wavelength range of 480 nm-630 nm, the wavelength at which the maximum absorption takes place is selected, and the absorption value of the supernatant in each well is read at the selected wavelength. Because the pre-sensitized recipient's lymphocytes have killing and damaging effect on the standard cell strain (target cells) containing donor antigens during the process of the proliferation culture. The more the target cells survive, the fewer the target cells are damaged. Because this method is used to determine the number of surviving target cell, the lower the light absorption value of a well is, the fewer the survival target cells in this well is, in other words, more target cells are killed and damaged. It also indicates that the antigens contained in this well are the donor antigens, which will cause rejection reaction in recipients. The average value of light absorption value of the wells with donor antigens is subtracted from that of the wells without donor antigens. The greater the resultant difference is, the more intensive the rejection reaction in the recipient is.

After the cells are cultured for 4-10 hours, an equivalent amount of organic solvent is added to each well of the cell assay plate to make formazane crystals in the damaged target cells dissolve. The supernatant in each well is harvested, respectively, and within the wavelength range of 480 nm-630 nm, the wavelength at which the maximum absorption takes place is selected, and the light absorption value of the supernatant in each well is read at the selected wavelength. This light absorption value can be used to determine if a rejection reaction has occurred in the body of the recipient. The mechanism thereof is substantially same as described above. The mentioned organic solvent should be able to dissolve formazane, but is not able to enter cells. For example, this solvent can be selected from the group consisting of methanol, ethanol, benzyl alcohol, formadehyde, acetaldehyde, glutaraldehyde, and dimethylbenzene.

The following cell assay plate can be used in the present method.

The used cell assay plate in the present invention is composed of the cell plate body and the cover. On the cell plate body, there are regularly set culture wells and the marking signs to locate every well. On the side directed towards well of the cover, there are sealing pads that correspond with each well. It is characterized by the structure as follows. The diameter of the culture well is 3-6 mm, and the deepness of the culture well is 5-15 mm. A protruded edge is set around outer peripheral sides of each well. The bottom of the well is flat and is connected with the peripheral wall of the well in a slanting fashion. Between the lower part of the inner side wall of the cover and its corresponding side wall of the cell plate body, a dual claming mechanism is set. This claming mechanism is a structure with transverse notches and flanges. When the cover is pressed over the cell plate body which make the flanges on two sides of the cover click into the first notch, respectively, the sealing pad on the cover doesn't seal the culture well completely. If the cover is further pressed downwards to make the flanges click into the second notch, the sealing pad will seal the culture well completely.

As seen from the above description, although most of the detection means in the present methods are conventional experiment methods in immunology, the invention discloses for the first time that a standard cell strain assay plate is used as a tool to detect the rejection reaction in the body of the recipient after organ transplantation utilizing the pre-sensitization theory. Unlike the known mixed lymphocyte reactions, the present invention takes advantage of the pre-sensitized lymphocytes of the recipient. Therefore, the stimulating reaction become a recalling reaction, which greatly reduces the reaction time. The known pre-sensitized lymphocytes-typing test uses the pre-sensitized lymphocytes prepared in vitro to test if the donor or recipient's cells contain certain antigen. But this invention uses the recipient's lymphocytes as pre-sensitized reaction cells, and uses standard cell strain as irritation cells to determine the immune status in the body of the recipient. At a glance, they appear to be similar, however, the used tool, the object and the purpose in both methods are very different. Unlike the known lymphocyte killer test, this invention uses the standard cell strain as the target cells, and makes it unnecessary for a donor to provide target cell source and thereby reduces the trouble in preparation. This invention takes adavantage of the conventional mixed lymphocyte reaction and pre-sensitized cell-typing test, and takes a shorter time accomplish the detection. The present invention can reduces the time necessary for the detection from 3 days of the conventional methods to less than 30 hours. The shortest time for obtaining the results with the present invention is just within 4-5 hours. In addition, for the present method, it is unnecessary for the donor to provide the target cells or irritation cells. Thus, neither the survival of the donor nor the source of the target cells nor irritation cells is necessary to be considered. Furthermore, even if the data regarding the types of the antigens of the donor is not available, it doesn't matter to the detection. The invention also inaugurate a new field for the application of the standard cell assay plate, and provide a means to dynamically monitor the immune status of the body of the recipient after organ transplantation. Therefore, based on the invention, the clinical doctors can predict the occurrence of rejection reaction before the symptoms and signs are appeared. The present invention can also provides a basis for adjusting the clinical immunosuppressant dosage administrated to the recipient, and increase the success rate of organ transplantation greatly. Compared with the known test methods, the present method also possesses the advantages of painless detection, shorter time, lower cost, higher specificity, and higher reliability.

### BRIEF DESCRIPTION OF THE DRAWINGS

FIG. 1 is a diagrammatic sketch showing the morphological features of lymphocyte transformation: “1” shows the morphological features of a lymphocyte wherein the said transformation doesn't take place; “2” shows the morphological features of a lymphocyte in the transitional state of transformation; and “3” shows the morphological features of the lymphoblast.

FIG. 2 is a front view of the cell assay plate (without the cover).

FIG. 3 is a side sectional view of the cell assay plate (with the cover). In FIG. 3, “4” is the cell assay plate body, “5” is the culture well, “6” is the signs for locating the culture wells, “7” is the cover of the cell assay plate, “8” is the flange, “9” is a the sealing pad, and “10” is the notche.

Hereinafter, in combination with the Drawings, the embodies and the technical effects of the cell culture determination plate will be described.

Referring to FIG. 3, it is a embody of the cell (culture) assay plate of the present invention. The cell assay plate body can be made of transparent glass, organic glass, or plastic through forming in one step, while the cover can be made of organic glass or plastic. The cover, the scaling pads and the flanges (or the notches) can be made through forming in one step, or the sealing pads and the flanges made of rubber can be bonded thereon. The culture wells were arranged into a regular array. The capacity of the culture well should be in the range of 0.05-0.5 ml, which varies depending on practical case of the assay. The plates with different capacity can be produced. The bottom of the culture well is flat, and is connected to the peripheral walls in a slanting fashion to eliminate the edging angles of the bottom so that the cells can be spread and washing is convenient. A protruded edge is set around outer peripheral side of each well (which is not shown in the Drawings) to prevent the liquid on the surface of the plate from flowing into the well and also make it more sealed after the cover is applied tightly. It can be made together with the cell plate body in one-step forming. Two notches were set transversely on both lateral walls of plate body corresponding to the lower part of the inner lateral wall of the cover. The notches match the transverse flanges on the lower part of the inner lateral wall of the cover. When the cover is pressed downward, the flanges can be clicked into the first notches on the plate body. At this time, there is a gap between the sealing pads and the culture wells to allow air exchange when cell culture is conducted. When the cover is pressed further downward, the flanges were clicked into the second notches, at which the sealing pads can seal the culture wells completely. It can be seen that if the flange is set on the plate body, and the both notches are located on the inner lateral sides of the downside of the cover, its usage will be more convenient. The cell assay plate designed in the present invention makes the application thereof more convenient.

As learned from the above described various detection methods, the present invention comprises different assay methods to monitor the immune status of the recipient's body after organ transplantation with the same principle of immunology but different detecting means for different reactive objects at different stages. Specifically, in the methods of the present invention, the standard cell strain is used as the target cells (irritation cells), and the recipient's lymphocytes are used as pre-sensitized effector cells (reaction cells). The standard cell strains on the cell assay plate contains almost all the known HLA antigens that can cause rejection reaction, and if there is any occurrence of a rejection reaction in the body of the recipient, then the recipient's lymphocytes will be turned into pre-sensitized memory cells. Therefore, when they meet the standard cell strains containing donor antigens during in vitro assay, the lymphocytes will present a recalling reaction immediately after being stimulated. On one hand, the lymphocytes will be turned into lymphoblast in large numbers to proliferate (which can be confirmed by determining cell proliferation with such methods as morphological observation, DNA labeling or MTT assay). On the other hand, the standard cell strain as the target cells will be challenged and damaged by the lymphoblast (which can be confirmed by such methods as dehydrogenase release test, or MTT assay on the standard cell strain treated with MTT during the process). This is a principal protocols of the present invention. The process of the various detection methods of the present invention will be illustrated in detail by the following examples.

In the following examples, in order to save material, some discarded materials in some examples is reused in other examples. The reuse and the necessary treatment therefor should not be considered as the necessary steps of these determination methods unless this reuse is proved to be necessary in the actual clinical test.

### EXAMPLES

### Example 1

### Preparation of the Standard Cell Strain Assay Plate

Although the standard cell strain assay plate produced by Lamda Co. contains various HLA antigens, they can't be directly used in this invention because the culture wells are too small in size and the number of cells is less. The cell assay plates designed according to this invention (64 wells, Diameter: 5 mm, Depth: 15 mm), 60 kinds of standard cell strains produced by Lamda Co. were commercially available and were prepared into the standard cell strain assay plate according to this methods of preparation. There are 40,000 cells in each well (this invention allows each well to contain 20,000-200,000 cells). The remaining 4 wells are added only with the standard cells as the maximum release control group of example 4.

### Example 2

### Morphological Observation of Reaction Cells

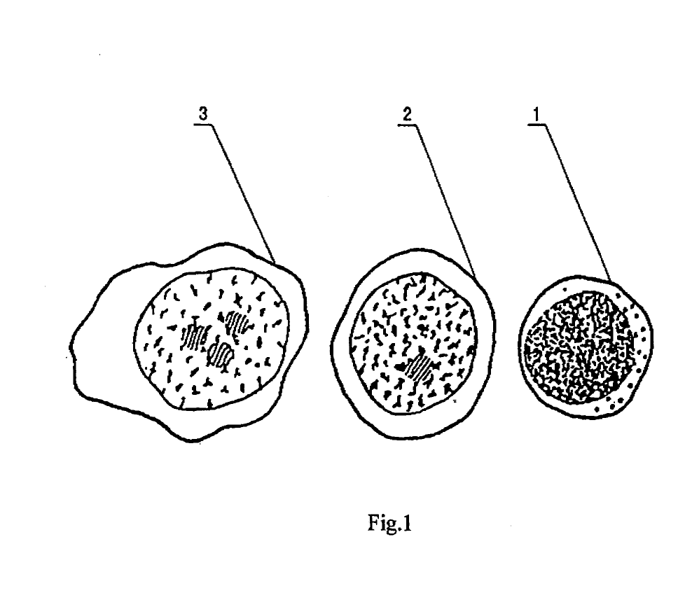

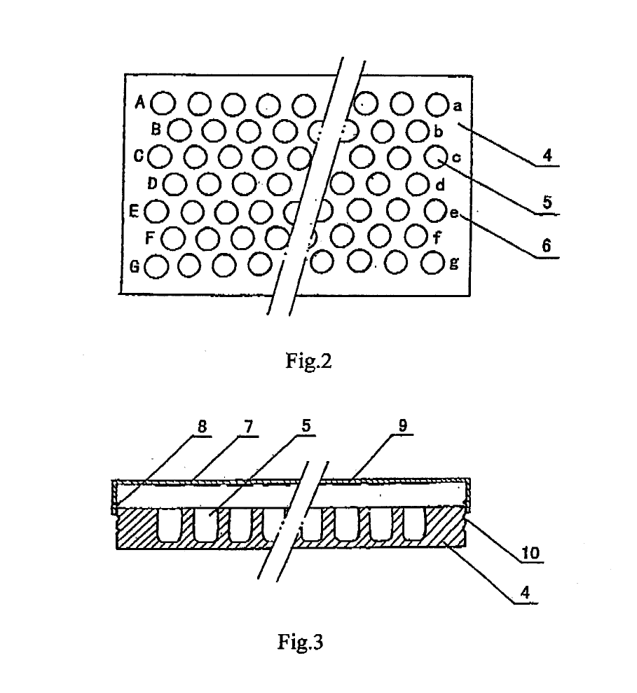

After organ transplantation, T-lymphocytes were separated from the recipient's peripheral blood and then were prepared into a cell suspension of 1 million cells per milliliter using RPMI 1640 culture medium containing 20% human AB serum (or DMEM culture medium). Then, to each well of the cell assay plate prepared in Example 1, 0.1 ml of the cell suspension was added (the target cells-the effector cells ratio is 1:2.5). The cell assay plate was placed into a CO₂ incubator with 5% of CO₂, and cultured at 36-38° C. for 2-28 hours. During this period, a microscope was used to observe if there is cell transformation at random time. The morphological features of cell transformation were shown in FIG. 1. If necessary, the cell counting can be conducted as disclosed by Experimental Technologies in Modern Immunology, edited by Guanxin Shen etc. (Hubei Science and Technology Press, China, October, 1998).

### Example 3

### Morphological Observation of Labeled Cells

After organ transplantation, T-lymphocytes were separated from the recipient's peripheral blood and then were prepared into a cell suspension of 400,000 cells per milliliter using the serum-free culture medium containing 0.2 mmol of fluorescence labeled TTP. Then, to each well of the cell assay plate prepared in Example 1, 0.1 ml of the cell suspension was added (the target cells-the effector cells ratio is 1:1). The cell assay plate was placed into a CO₂ incubator with 5% of CO₂ , and cultured at 37° C. for 10-28 hours. During this period, a fluorescence microscope was used to observe if there is fluorescence in the cell nucleus. Although there are various labels and labeling methods nowadays, the present invention only needs the labeled signals obtained by direct morphological observation, such as ferritin-labeling and colloidal gold labeling (that can make nucleus become black), fluorescein labeling and luminous material labeling (that can make nucleus luminous).

### Example 4

### Detection of Dehydrogenase Release from the Target Cells

Using the cell assay plate that has been incubated for 2 hours in Example 2, 0.1 ml of 1% NP-40 (non-ionic detergent) solution was added to 4 control well only with the standard cells. Then the cell plate was cultured for additional 2 hours and was taken out. 0.1 ml of the supernatant in each well was drawn out and was placed into the corresponding culture wells in another blank assay plate (no cell strain), respectively. 0.1 ml of the fresh prepared substrate solution was added to each well and allowed to react for 15 minutes at the room temperature under keeping off light. Then, 30 μl of citric acid stop solution (1 mol/L) to stop the reaction. The light absorption value in each well was read with ELISA reader (a product of Eastern China Electron Tube Factory) at the wavelength of 570 nm. The average light absorption value of the wells without donor antigens was selected as the light absorption value of the natural release control group, and the calculation was conducted according to the method above described previously. The present method can be used to determine not only the release of a dehydrogenase (lactate dehydrogenase, or malate dehydrogenase, or glutamate dehydrogenase), but also the release of various kinds of dehydrogenase, provided that the reaction substrates thereof should be contained in the substrate solution. These reaction substrates can be any one or more than one selected from the group consisting of sodium lactate, sodium malate, or sodium glutamate and their combinations. This example employed the LDH substrate solution (containing sodium lactate), and the preparation of the substrate solution and the procedure of enzymatic reaction were conducted as found on page 311 of the book Experimental Technologies in Modern Immunology.

### Example 5

### MTT Determination of Cell Transformation

Using the previous cell plate after the supernatant was removed in Example 4 (excluding the 4 wells of the maximum release control group), 0.1 ml of the culture medium was add into each well, then the plate was placed into the incubator to continue culture for 6 hours. Then the plate was taken out, and 10 μl of solution of MTT in PBS was added to each well (the concentration being 5 mg/ml), and the plate was again placed into the incubator to culture for 4 hours. The supernatant was drawn out from each well and the light absorption value thereof was read at the wavelength of 480 nm using the ELISA reader.

### Example 6

### MTT Determination of Cell Transformation

Using the previous cell plate after the supernatant was removed in Example 5, the remaining supernatant was removed completely in each well. 0.2 ml of DMSO was added to each well. The plate was shaken for 5 minutes. Then the light absorption value of the supernatant of each well was read at the wavelength of 490 nm using ELISA reader. In the present method, there is also a absorption peak at 630 nm. So, the light absorption value can be read at this wavelength too, or at both wavelengths. The specific calculation method and judgment were conducted according to the paper of “the Improvement of MTT Colorimetric Analysis and the Preliminary Applications, Shanghai Journal of Immunology, No.5, 1996).

### Example 7

### Preparation of Cell Assay Plate with the Standard Cell Containing Formazane Reduced from MTT

Just before the accomplishment of the standard cell strain culture, MTT was added to each cell culture medium in final concentration of 0.25 mg/ml. The culture was continued for 4 hours. The assay plate was prepared according to the original method. In the resultant cell assay plate, each cell contains the same amount of formazane. These cell plates can be obtained by treating the standard cell plate with MTT. For the treatment procedure on the standard cell strain with MTT in this example and MTT assay procedure was conducted according to the book Experimental Technologies in Modern Immunology.

### Example 8

### MTT Assay of the Target Cells

Using the cell assay plate prepared in Example 7, 0.1 ml of the recipient's lymphocyte suspension prepared in example 2 was added to each well (target-effector ratio is 1:2.5). The assay plate was placed into the incubator and cultured for 4 hours. Then, the assay plate was taken out and 20 μl hydrogen peroxide solution (concentration: 1%) was added to every well therein. Then the assay plate was placed back into the incubator and cultured for additional 2 hours and then was taken out again. The light absorption value of the supernatant in each well was read at the wavelength of 480 nm using ELISA reader. The colorless agent which can oxidize formazane crystal into MTT, has no affect on normal cells, was selected as the oxidizing agent in this example. There is no strict requirement on the added amount of the oxidizing agent and the concentration thereof, provided that it is enough to achieve the effect and its amount in every well is the equivalent.

### Example 9

### MTT Assay of the Target Cells

Using the previous cell plate after the supernatant was removed in Example 8, the remaining supernatant was removed completely in each well. 150 μl of DMSO was added to each well. The plate was shaken for 10 minutes. Then the light absorption value of the supernatant of each well was read at the wavelength of 630 nm using ELISA reader. The method for reading the assay plate and the calculation were same as Example 6.

### Example 10

### MTT Assay of the Target Cells

Using the cell assay plate prepared in Example 7, 0.1 ml of the lymphocyte suspension prepared in example 2 was added to each well (target-effector ratio is 1:3). The assay plate was placed into the incubator and cultured for 8 hours. Then, the assay plate was taken out and 0.1 ml of ethanol was added to every well therein. Then the assay plate was shaken for 5 minutes. The light absorption value of the supernatant in each well was read at the wavelength of 570 nm using ELISA reader. The method for reading the assay plate and the calculation were same as Example 6. The organic solvents that can replace ethanol is described hereinbefore.

### Example 11

### Assay of Tolerance

The reagents in the cell assay plates in Examples 2, 3, 4, 5, 6, 8, 9, and 10 were washed away (retaining the cells). 0.2 ml of culture medium was supplemented. Then the cell assay plates was placed into the incubator to incubate for 3-5 days, at which the cell assay plates were taken out and to each well, 20 μl of MTT solution (5 mg/ml) was added, and continue to culture for 2-6 hours. The supernatant was removed. Then 0.2 ml of DMSO was added to every well. The light absorption value of the supernatant in each well was read at the wavelength of 490 nm or 630 nm, respectively. If the light absorption value of the wells containing donor antigens is significantly lower than that of the wells without donor antigens, then it shows that the recipient has already developed tolerance to the donor's organ. Thus the immunosuppressant administrating to the recipient can be reduced in dosage or be stopped under regular monitor with the method of the present invention. If the light absorption value of the wells containing donor antigens is significantly higher than that of the wells without donor antigens, then it shows that there is an acute rejection reaction (when being consistent with the assay results within 30 hours above mentioned) or chronic rejection reaction (when being inconsistent with the assay results within 30 hours above mentioned).

With a lot of experiments, the inventor of this invention believes that in various assay methods of this invention, the time length of cell culture is related to the sensitization degree of the lymphocytes. When the cells are highly sensitized (acute rejection reaction), a short time of culture can result in response in an assay. However, when the cells are poorly sensitized (at the initial stage of rejection reaction), a longer period of culture is needed in an assay. In the practical detection, this can be adjusted based on the clinical experience. Although the data obtained by the methods of the invention can used to determine if there is rejection in the body of the recipient and how serious it is, it can't used to quantify the rejection reaction directly. The extent of rejection reaction can be quantified by analysing the recipient's all assay results and the operator's experience. In practically clinical application of this invention, if no experience, the operator had better use two parallel methods for assay so that the assay results can be compared and confirmed. That is to say, the operator can select two different methods (one for assay of the effector cells, and the other for target cells) from the present invention to assay at the same time. After accumulation of rich experience, one method is enough.

The specificity of this invention lies in the fact that a single standard cell assay plate can not only determine the specific killer activity of CD8⁺ T-lymphocytes and NK cells from the recipient towards donor cells, but also determine the specific transformation and proliferation ability of CD4⁺ T-lymphocytes from the recipient under the stimulation of donor antigens. In the assay methods, not only a number of wells containing donor antigens can be mutually used as control to be compared from each other, but also the specific killer activity of CD8⁺ subgroup and the specific proliferation ability of CD4⁺ subgroup can be used as controls between both experimental methods. These make the present methods not only specific and sensitive, but also reliable for monitoring the rejection reaction.

The present invention can not only be used to monitor the immune status after allogenic organ transplantation, but also be used to monitor the immune status after heterogenous organ transplantation, provided that the standard cell strain containing various animal antigens is available.

There are many methods to prepare the standard cell strains as described in the present invention. The following methods can be used with genetic engineering technology. A cell strain such as B lymphocyte strain was selected as the object for modification. The HLA gene fragment on its chromosome was removed with the method of gene knockout. Then, according to homologous gene recombination theory, a gene of certain type of HLA antigens was transfected into the cell so that a standard cell strain that only expresses one Type I or one Type II HLA antigens can be developed.

The specific method for preparing the standard cell strain utilizing genetic engineering technology is as follows. A human B-lymphocyte strain may be selected as the object for modification. The nucleotide sequence of human HLA gene was found out from the GenBank, the primers were designed so that two 2-4 kb DNA fragment on two ends of HLA gene were amplified, respectively, as the homologous recombination region. The resultant upstream and downstream fragments of the HLA gene were purified for use (Cell, 1991, 66:1051-1066; Science, 1982, 256:1392-1394). A DNA expression vector of eukaryocyte (so called eukaryotic expression vector, such as Living Color™ Fluorescein Protein Reporting System, SV40 vector, etc) with a antibiotic screening marker gene (such as neomycin resistance gene (neo), hygromycin resistance gene (HYP), puromycin resistance gene, Zeocin, etc) was selected to construct HLA gene knockout vector using conventional genetic engineering approaches. That is to say, a tandem structure consisting of marker gene located between upstream fragment and downstream fragment was inserted into the selected eukaryotic expression vector. Then, the established gene knockout vector was propagated with conventional molecular biological approaches.

After extraction by the conventional methods, the purified gene knockout vector is transfected into the selected B cell strain with electroporation or other molecular biological methods for cell culture. At the same time, the antibiotic corresponding with the selected resistance gene was added in order to screen the cell strain with resistance gene (with resistance gene, indicating that the target gene has been transfected into the cell strain). Then, the genome DNA is extracted from the screened cell clones and was identified by polymerase-chain reaction (PCR) with the primers specific to HLA gene. The cell strain whose both HLA alleles in the homologous chromosomes have been replaced was selected as blank cell strain that doesn't express human HLA for future use (This cell strain doesn't contain HLA antigen gene, that is to say, the normal human HLA genes have been knocked out from this cell strain). Then, the cDNAs or genome DNAs of almost all HLA antigens were are prepared by reverse transcription or screening out from the human genome library, respectively. Then, they were introduced into the above mentioned expression vector (switching a resistance gene). Then, according to the above mentioned methods, the expression vectors, carrying different HLA antigen gene, respectively, were introduced into the above mentioned blank cells (one gene, one cell strain) in which genes of HLA antigens had been knocked out. This is to say, cell strains that expresses various HLA antigens, respectively, were obtained, i.e. standard cell strains. Then, a system of standard cell strain that expresses various HLA antigens respectively is established.

What is claimed is

1. A method for determining the reactivity of lymphocytes in blood to a specific antigen(s), comprising:
the T-lymphocytes in the peripheral blood of the recipient are prepared into a cell suspension with cell culture medium;
using a cell assay plate prepared with the standard cell strains containing the known HLA antigens, the cell suspension is added to each well of the cell assay plate in the target-effector ratio of 1:0.5-10;
the cells are cultured for 2-28 hours; and
then, the difference between the reactivity of the recipient's lymphocytes in the well of the cell strain containing donor antigens and that of the lymphocytes in other control wells containing no donor antigen is distinguished, and thereby, whether the recipient's lymphocytes are sensitized can be determined.

2. A method as claimed in claim 1, wherein the said detection is conducted by observing the presence of lymphocyte transformation in each well using a microscope, and obtaining the transformation rate by cell number counting.

3. A method as claimed in claim 1, wherein the said culture medium is a culture medium containing labeled TTP, and during the 6-28 hours for cell culture, the labeled TTP is incorporated in the synthesis of intracellular DNA, which make the cell nucleus present directly certain significant feature under common microscope or fluorescence microscope.

4. A method as claimed in claim 1, wherein, after cell culture for 2-6 hours, a equivalent amount of the supernatant is removed from each well into the corresponding culture well of another blank assay plate, respectively, in order to carry out enzymatic reaction of dehydrogenase assay, and after the reaction is stopped, within the wavelength range of 480 nm-630 nm, the wavelength at which the maximum absorption value takes place is selected and the light absorption value of the solution in each well is read at the selected wavelength.

5. A method as claimed in claim 1, wherein, after the cell culture for 6-16 hours, a equivalent amount of 3-(4,5-dimethylthiazol-2-yl)-2,5-diphenyltertra-zoliumbromide (MTT) solution is added to each well of the cell assay plate, respectively, and the cell culture is continued for 2-6 hours, and after the culture is stopped, the supernatant in each well is harvested, and within the wavelength range of 480 nm-630 nm, the wavelength at which the maximum absorption of MTT solution takes place is selected, and the light absorption value of the solution in each well is read at the selected wavelength.

6. A method as claimed in claim 5, wherein, after the culture is stopped, to each well of the cell assay plate in which the supernatant has been removed, an equivalent amount of DMSO is added so that intracellular formazane crystal is dissolved out, and within the wavelength range of 480 nm-630 nm, the wavelength with the maximum absorption value is selected, and the light absorption value of the supernatant in each well is read at the selected wavelength.

7. A method as claimed in claim 1, wherein each standard cell strain used to prepare the cell assay plate contains an equivalent amount of formazane crystals from the reduction of MTT. After cell culture as above, MTT assay can be used to detect MTT or formazane crystals in each well.

8. A method as claimed in claim 7, wherein the said MTT assay is as follows: after the cells are cultured for 3-8 hours, an equivalent amount of oxidizing agent is added to each well of the cell assay plate, the culture continues for 1-4 hours, the oxidizing agent can oxidize formazane crystals in the damaged target cells into MTT, and after the cell culture is stopped, within the wavelength range of 480 nm-630 nm, the wavelength at which the maximum absorption value of the MTT solution takes place is selected and the light absorption value of the supernatant in each well is read at the selected wavelength.

9. A method as claimed in claim 7, wherein the said MTT assay is as follows: after the cells are cultured for 4-10 hours, the supernatant is removed, an equivalent amount of DMSO solution is added to each well to make formazane crystals in intact target cells be dissolved out, and then, the supernatant in each well is harvested and within the wavelength range of 480 nm-630 nm, the wavelength at which the maximum absorption takes place is selected, and the light absorption value of the supernatant in each well is read at the selected wavelength.

10. A method as claimed in claim 7, wherein the said MTT assay is as follows: after the cells are cultured for 4-10 hours, an equivalent amount of organic solvent is added to each well of the cell assay plate to make formazane crystals in the damaged target cells be dissolved, the supernatant in each well is harvested and within the wavelength range of 480 nm-630 nm, the wavelength at which the maximum absorption takes place is selected, and the light absorption value of the supernatant in each well is read at the selected wavelength.

11. A method as claimed in claim 10, wherein the said organic solvent is selected from the group consisting of methanol, ethanol, benzyl alcohol, formadehyde, acetaldehyde, glutaraldehyde, and dimethylbenzene.

12. A method as claimed in any one of claims 1-11, wherein the structure of the cell assay plate is as follows: the diameter of the culture well therein is 3-6 mm, and the deepness of the culture well is 5-15 mm, a protruded edges is set around outer peripheral sides of each well, the bottom of the well is flat and is connected with the peripheral wall of the well in a slanting fashion, and a dual claming mechanism is set between the lower part of the inner side wall of the cover and its corresponding side wall of the cell plate body, which is a structure of transverse notches-flanges, when the cover is pressed downward on the cell plate body which make the flanges on two sides click into the first notch, respectively, the sealing pad on the cover can not seal the culture well completely, but if the cover is further pressed downwards to make the flanges click into the second notch, the sealing pad will seal the culture well completely.

13. A method as claimed in any one of claims 1-11, wherein the standard cell strain is prepared as follows:
B lymphocyte strain is selected as the object for modification, the HLA gene fragment on its chromosome DNA was removed with the method of gene knockout;
then, a gene of a specific HLA antigen is inserted into its chromosome DNA and allowed to be expressed so that a standard cell strain system that expresses only one HLA antigen can be developed.
